(19) United States
(12) Patent Application Publication
(10) Pub. No.: US 2009/0105197 A1
(43) Pub. Date: Apr. 23, 2009

(54) Use of Sodium Channel Blocking Compounds and Aspirin in Manufacturing Drugs for Producing Analgesia Synergistically in Mammals

(75) Inventors: Baoshan Ku, Beijing (CN); Hay Kong Shum, North Point (HK)

Correspondence Address: FISH & RICHARDSON PC, P.O. BOX 1022, MINNEAPOLIS, MN, 55440-1022, US

(73) Assignee: WEX MEDICAL LIMITED, Hong Kong (HK)

(21) Appl. No.: 12/249,802

(22) Filed: Oct. 10, 2008

Related U.S. Application Data
(62) Division of application No. 10/480,288, filed on Apr. 1, 2004, now abandoned.

(30) Foreign Application Priority Data
Jun. 22, 2001 (CN) 01115990.1

(51) Int. Cl. A61K 31/60; A61P 29/00

(52) U.S. Cl. 514/161; 514/165

(57) ABSTRACT

This invention relates to the use of combinations of a sodium channel blocking compound that binds to an SSI or SS2 site of extracellular region of a sodium channel alpha subunit, and aspirin in manufacturing drugs for producing synergistically analgesic effect in mammals. Pharmaceutical compositions based upon this invention can enhance analgesic effect and reduce dosage of aspirin, therefore side effects and adverse reactions are decreased accordingly.

This application is a divisional of U.S. application Ser. No. 10/480,288, filed Dec. 11, 2003, the national phase under 35 U.S.C. § 371 of PCT International Application No. PCT/CN02/00428, which has an International filing date of Jun. 18, 2002, designated the United States of America, and claims the benefit of priority of Chinese Application 01115990.1, filed Jun. 22, 2001. U.S. application Ser. No. 10/480,288 is hereby incorporated by reference in its entirety.

This invention relates to the use of combinations of a sodium channel blocking compound that binds to an SSI or SS2 site of extracellular region of a sodium channel alpha subunit, and aspirin in manufacturing drugs for producing synergistically analgesic effect in mammals. Pharmaceutical compositions based upon this invention can enhance analgesic effect and reduce dosage of aspirin, therefore side effects and adverse reactions are decreased accordingly.

Pharmacologically, anti-inflammation drugs consist of two major kinds: aspirins and steroids. Aspirin is a very widely used non-steroid analgesic, as well as an anti-inflammatory analgesic. Belonging to the category of acetylsalicylic acids, aspirins mainly comprise acetylsalicylic acids (commonly known as aspirin), salicylates (mainly sodium salicylates) and diflunisal₁. Salicylic acid is the active ingredient in a salicylate. ¹Diflunisal is a difluorophenyl derivative of salicylic acid.—trans

Inhibition of prostaglandin (PG) synthesis is the major mechanism of action for aspirin-alike drugs to produce pharmacological, therapeutic, and toxic and side effects. Aspirin has such effects as inhibiting synthesis of pain sensation exciting substances like bradykinin and histamine, restraining activity of white blood cells, influencing the body temperature adjusting center in the hypothalamus, thereby producing analgesic, anti-inflammatory and antipyretic effects. Aspirin also impairs thromboxane (TXAT) synthesis by inhibiting prostaglandin cyclooxygenase in platelets, thereby inhibiting platelet aggregation. (Xiaozhi CHENG, Pingtian XIAO, Zhongshen WANG, New Edition of Practical Manual for Drugs, 1994, SS10034400, Chaoxing Digital Library).

Aspirin has a remarkable analgesic effect in alleviating pain caused by common cold, as well as treating headache and fever induced by general mental stress. It is used mainly for treatment of the following indications:
1. Common cold, fever, mild to moderate pain (headache, dental pain, neuromuscular pain, menstrual pain et cetera.);
2. Rheumatism, rheumatic joint arthritis;
3. Generation of thrombus. Regimen of small doses for long term is necessary.

Aspirin may cause side effects as following (Qingwei SUN, Yi HOU, Novel Clinical Uses of Aspirin-Alike Drugs and Adverse Effects, 1998, SS10034347, Chaoxing Digital Library):
1. Stomach pain, occasionally gastric ulcer and bleeding; asthma, skin rash in allergic reactions; occasionally reversible hepatic or renal damage.
2. Overdose reactions: mild reactions include salicylism; severe ones comprise hematuria, convulsion, hallucination, psychiatric disorder, and difficulty in respiration.
3. Long term use of aspirin is associated with false positive results in examination of sugar in urine, false escalation in serum uric acid, abnormal level of transaminase, decrease in cholesterol, hypokalemia, and prolonged thrombinogenesis.

In 1990s, aspirin was found to have statistically significant effect on preventing stroke and heart diseases in middle-aged people if it was taken frequently. Apparently, aspirin possesses such mild anti-coagulation property that it prevents blood clots, thereby improving blood circulation.

Aspirin is inexpensive while delivering sound therapeutic effects with minor adverse reactions, so it is widely used as an over-the-counter drug. However, aspirin at large doses at some occasions, particularly when needed to produce desirable such therapeutic effects as alleviating refractive pain induced by rheumatism and arthritis, could cause gastric ulcer, ischemia, or bleeding in the upper gastrointestinal tract. Although the bleeding is not of big amount, it will become a serious problem if aspirin is taken at large doses for a continuous period. Especially when there has been ailment in the gastrointestinal tracts, overdose of aspirin could even cause death, or at least intoxication symptoms like ulcer, gastric dilatation, and thinned anterior gastric branches. In U.S. Pat. No. 4,491,574, Seifer et al provided in 1985 a solution for diminishing intoxication of aspirin by taking vitamin A simultaneously or in advance so as to increase gastric secretion. This invention discloses an alternative approach, which is to reduce dosage of aspirin by combining a synergistic analgesic in case large doses of aspirin are required for producing analgesia.

Tetrodotoxin (TTX) is a potent non-protein neurotoxin possessing pharmacological effects like analgesia, local anesthesia and anti-convulsion. TTX noticeably alleviates various types of dull pain and sharp pain, and does not induce dependence. However, its value for clinical application is limited by dosage. From the perspective of practicality, the synergistic interaction between drugs is studied. In order to measure the probability of using TTX as a synergistic analgesic clinically, a chemical stimulation model, namely acetic acid induced writhing test in mice (sensitive to antipyretic analgesics) was employed to observe the interaction between small doses of TTX and aspirin, an antipyretic and analgesic drug.

The mechanism of action for TTX to produce analgesia is to inhibit the generation and transmission of neuron pulse by blocking the TTX-sensitive (TTX-S) sodium channels thus the inward sodium current. Aspirin as an antipyretic and analgesic drug inhibits cyclooxygenase so as to impair synthesis of prostaglandin (PG) and to depress the pain-inducing and hyperalgesic effect of prostaglandin (PGE2), and alleviate bradykinin's pain-inducing effect as well.

It has been disclosed in literature (Cesare P, Mcnaughton P, Peripheral pain mechanisms. Curr Opin Neurobiol 7(4):493-9, 1997 August) that hyperalgesia caused by tissue injury is related to two mechanisms at the least: sodium current induced by bradykinin which increases thermal irritation, and gate voltages of several types of PGE2-influenced ion channels including TTX-resistant sodium channels. The hyperalgesia effect of PGE2 is related to TTX-R sodium channels (Knasar S G; Gold M S; Levine J D. A tetrodotoxin-resistant sodium current mediates inflammatory pain in the rat. Neurosci Lett, 256(1):17-20, 1998 Oct. 30), as PGE2 raises the amplitude of TTX-resistant sodium current (TTX-RINa), thereby enhances the activity of TTX-R sodium channel. Under the circumstances of chronic pain, the sensitization of nociceptors are mediated through TTX-R sodium channels (Tanaka M; Cummins T R: Ishikawa K; Dib-Hajj S D; Black J A; Waxman SQSNS Na+ channel expression increases in dorsal root ganglion neurons in the carrageenan in flammatory pain model. Neuroreport, 9(6):967-72 1998 Apr. 20), (Krai M G; Xiong Z; Study R E, Alteration of Na+ currents in dorsal root ganglion neurons from rats with a painful neuropathy. Pain 81(1-2): 15-24 1999 May). Therefore, analgesia effect can be produced by blocking TTX-R sodium channels (Akopian A N; Souslova V; England S; Okuse K; Ogata N; Ure J; Smith A; Kerr B J; McMahon S B; Boyee S; Hill R; Stanfa L C; Dickenson A H; Wood J N. The tetrodotoxin-resistant sodium channel SNS has a specialized function in pain pathways. Nat Neurosci, 2(6):541-8 1999 June). This explains that the analgesic effect of TTX did not increase markedly with escalating doses in a previous heat-induced tail flick test in mice. Aspirin induces analgesia by impairing synthesis of PGE2 and thus decreasing the sodium current conveyed by TTX-R channels. Therefore, synergistic analgesia by combining aspirin and TTX is hypothesized as they may jointly inhibit TTX-sensitive and TTX-resistant sodium channels simultaneously.

The acetic acid-induced writhing in mice indicated that tetrodotoxin yielded 40.6% and 27.7% inhibition at doses of 1/25 and 1/50 LD50 (0.79 μg/kg, 0.39 μg/kg), respectively, which was in accordance with the literature. When combined with aspirin, tetrodotoxin at the two dose levels reduced the half inhibition dose (ID50) of aspirin from 44.1 mg/kg alone to 5.0 mg/kg, 10.0 mg/kg, and the 95% inhibition dose (ID95) from 361.8 mg/kg alone to 94.5 mg/kg, 154.3 mg/kg, respectively. Isobolographic analysis proved that there was significant synergistic interaction between aspirin and TTX.

This invention is meaningful in that it provides a novel approach for treating pain clinically, particularly some types of acute and chronic pain which do not respond well to current antipyretic analgesics, by using small doses of tetrodotoxin in combination so as to improve analgesic effect and reduce dosage of involved drugs, thereby reducing adverse reactions.

### EXAMPLE

### 1 Materials and Methods

### 1.1 Animals

Kunming mice, 18-22 grams, half male and half female, supplied by the Experimental Animal Center of Beijing University, Medical Branch. Quality Certificate No. 013056. Classification: One.

### 1.2 Test Article and Reagents

Tetrodotoxin (TTX), 95% purity, supplied by Nanning Maple Leaf Pharmaceutical Co., LTD., batch no. 0324C. Diluted with citric acid buffer solution to required concentration. Aspirin (ASP), powder, 99% purity, manufactured by Shandong Xinhua Pharmaceutical Factory, batch no. 0005564. Ground and then diluted with 0.5% sodium carboxymethyl cellulose (CMC) solution. Glacial acetic acid, analytical pure, manufactured by Beijing 52952 Chemical Factory, batch no. 991117.

### 1.3 Methods

Acetic acid-induced writhing test in mice (Shuyun X U, Rulian BIAN, Xiu CHEN, Methodology of Pharmacology Experiments). 380 mice were selected, given no food but drinking water 12 hours prior to the experiment, randomly divided into 19 groups: control group (CMC solution), solely ASP groups (25 mg/kg, 50 mg/kg, 100 mg/kg, 150 mg/kg, 200 mg/kg, totally five groups), solely TTX groups ( 1/25 and 1/50 LD50 doses, or 0.79, 0.39 ug/kg, respectively), and combined groups: TTX (0.39 ug/kg)+ASP(6 mg/kg, 12.5 mg/kg, 25 mg/kg, 50 mg/kg, 75 mg/kg), TTX (0.79 ug/kg)+ASP(3 mg/kg, 6 mg/kg, 12.5 mg/kg, 25 mg/kg, 50 mg/kg, 75 mg/kg). Solely TTX or ASP was given to mice intramuscularly. For combined groups, drugs were given to both sides of a mouse intramuscularly at a volume of 0.1 mL/10 g, respectively. After 40 minutes, 0.6% glacial acetic acid solution was given intraperitoneally to induce pain. In the following 15 minutes, writhing movements were observed and recorded. Sign of a writhing movement was recognized to be positive when a mouse manifested repeated contraction of lumbar muscle, inward contraction of stomach, stretch of trunk and hind limbs, upward movement of buttock. The writhing inhibition rate was calculated according to the following formula:

Inhibition rate (%)=(the writhing incidences in the control group−those of a test group)/the writhing incidences in the control group×100%

The half inhibition rate (IDso) was determined by the probit method.

### 1.4 Statistical Analysis

The SPSS software was employed for the statistical analysis, while the isobolographic analysis was performed to test the drug-drug interaction. (Duanzheng X U, Application of Biostatistics in Pharmacology, Science Publishing, 1986, 357-359), (Shuqin YANG, Medical Statistics, Encyclopedia of Chinese Medical Sciences, Shanghai Science and Technology Publishing, 1985, 197).

### 2. Results

As shown in Table 1, aspirin alone had a half inhibition dose (ID50) of 44.11 mg/kg in the acetic acid-induced writhing assay in mice. Combined with small doses of TTX ( 1/25 and 1/50 LD50), aspirin had its ID50 reduced to 5.01 mg/kg and 96 mg/kg, respectively, and ID95 reduced to 94.47 mg/kg, 154.33 mg/kg from 361.77 mg/kg, respectively. The reduction of both ID50 and ID95 exceeded two folds.

TABLE 1
Synergistic Action between TTX and Aspirin (i.m.) by Mouse Writhing Assay

| | | | Average | | ID₅₀ and 95% | | ID₉₅ and | |
|---|---|---|---|---|---|---|---|---|
| | Doses | No. of | Writhing | inhibition | Confidence Interval | | 95% Confidence | |
| Groups | (mg/kg) | animals | movements | Rate (%) | (mg/kg) | | Interval (mg/kg) | |
| Sodium carboxymethyl | 50 | 20 | 39.0 ± 15.4 | — | | | | |
| cellulose solution, control | | | | | | | | |
| TTX | 0.79 × 10⁻³ | 20 | 23.2 ± 11.7 | 40.6 | | | | |
| | 0.39 × 10⁻³ | 20 | 28.2 ± 9.65 | 27.7 | | | | |
| ASP | 25 | 20 | 25.1 ± 14.5 | 35.6 | | | | |
| | 50 | 20 | 19.3 ± 13.8 | 50.6 | 44.1 | (24.9~61.9) | 361.8 | (197.3~1689.2) |
| | 100 | 20 | 12.0 ± 9.2 | 69.3 | | | | |
| | 150 | 20 | 5.2 ± 5.7 | 86.6 | | | | |
| | 200 | 20 | 2.7 ± 1.9 | 93.2 | | | | |
| TTX(0.79 μg/kg) + ASP | 3.0 | 20 | 23.0 ± 8.3 | 40.9 | | | | |
| | 6.0 | 20 | 20.3 ± 12.2 | 47.9 | 5.0 | (3.8~6.3) | 94.5 | (62.7~170.7) |
| | 12.5 | 20 | 9.5 ± 9.0 | 75.7 | | | | |
| | 25.0 | 20 | 7.2 ± 6.9 | 81.6 | | | | |
| | 50.0 | 20 | 4.0 ± 4.6 | 89.9 | | | | |
| | 75.0 | 20 | 1.8 ± 1.2 | 95.4 | | | | |
| TTX(0.39 μg/kg) + ASP | 6.0 | 20 | 25.0 ± 10.9 | 35.8 | | | | |
| | 12.5 | 20 | 17.0 ± 8.9 | 56.4 | 10.0 | (7.6~12.3) | 154.3 | (99.8~301.1) |
| | 25.0 | 20 | 10.0 ± 11.1 | 74.5 | | | | |
| | 50.0 | 20 | 5.8 ± 5.3 | 85.2 | | | | |
| | 75.0 | 20 | 4.5 ± 3.3 | 88.6 | | | | |

1-10. (canceled)

11. A method for producing analgesia in a subject experiencing pain, the method comprising:
a. administering to the subject at least one sodium channel blocking compound, wherein the sodium channel blocking compound is selected from the group consisting of tetrodotoxin, derivatives of tetrodotoxin, saxitoxin, derivatives of saxitoxin, and mixtures thereof; and
b. administering to the subject at least one aspirin-like compound;
wherein the administration of the sodium channel blocking compound and the aspirin-like compound to the subject is effective to produce analgesia in said subject.

12. The method of claim 11, wherein the aspirin-like compound is a salicylic acid or a derivative or salt thereof.

13. The method of claim 12, wherein the sodium channel blocking compound and the aspirin-like compound are co-administered.

14. The method of claim 13, wherein the co-administration of the sodium channel blocking compound and the aspirin-like compound results in a synergistic effect whereby improved analgesia is achieved in said subject as compared to analgesic effect of either the sodium channel blocking compound or the aspirin-like compound administered in the absence of the other compound.

15. The method of claim 14, wherein the sodium channel blocking compound and the aspirin-like compound are each co-administered using the same route of administration.

16. The method of claim 15, wherein the route of administration is by injection.

17. The method of claim 16, wherein injection is an intramuscular injection.

18. The method of claim 14, wherein the subject is experiencing acute pain.

19. The method of claim 14, wherein the subject is experiencing chronic pain.

20. The method of claim 11 in which the at least one sodium channel blocking compound is selected from the group consisting of tetrodotoxin, dehydrotetrodotoxin, aminotetrodotoxin, methoxytetrodoxin, ethoxytetrodotoxin, deoxytetrodotoxin, tetrodonix acid, saxitoxin and mixtures thereof.

21. The method of claim 20, wherein the aspirin-like compound is a salicylic acid or a derivative or salt thereof.

22. The method of claim 21, wherein the co-administration of the sodium channel blocking compound and the aspirin-like compound results in a synergistic effect whereby improved analgesia is achieved in said subject as compared to analgesic effect of either the sodium channel blocking compound or the aspirin-like compound administered in the absence of the other compound.

23. The method of claim 22, wherein the sodium channel blocking compound and the aspirin-like compound are co-administered.

24. The method of claim 23, wherein the sodium channel blocking compound and the aspirin-like compound are each co-administered using the same route of administration.

25. The method of claim 24, wherein the route of administration is by injection.

26. The method of claim 25, wherein injection is an intramuscular injection.

27. The method of claim 22, wherein the subject is experiencing acute pain.

28. The method of claim 22, wherein the subject is experiencing chronic pain.

29. The method of claim 11, wherein the sodium channel blocking compound is tetrodotoxin.

30. The method of claim 29, wherein the aspirin-like compound is a salicylic acid or a derivative or salt thereof.

31. The method of claim 30, wherein the co-administration of the sodium channel blocking compound and the aspirin-like compound results in a synergistic effect whereby improved analgesia is achieved in said subject as compared to analgesic effect of either the sodium channel blocking compound or the aspirin-like compound administered in the absence of the other compound.

32. The method of claim 31, wherein the sodium channel blocking compound and the aspirin-like compound are co-administered.

33. The method of claim 32, wherein the sodium channel blocking compound and the aspirin-like compound are each co-administered using the same route of administration.

34. The method of claim 33, wherein the route of administration is by injection.

35. The method of claim 34, wherein injection is an intramuscular injection.

36. The method of claim 31, wherein the subject is experiencing acute pain.

37. The method of claim 31, wherein the subject is experiencing chronic pain.

38. The method of claim 11, wherein the aspirin-like compound is selected from the group consisting of salicylic acid or a salt thereof, acetylsalicylic acid, and diflunisal.

39. The method of claim 38, wherein the co-administration of the sodium channel blocking compound and the aspirin-like compound results in a synergistic effect whereby improved analgesia is achieved in said subject as compared to analgesic effect of either the sodium channel blocking compound or the aspirin-like compound administered in the absence of the other compound.

40. The method of claim 39, wherein the sodium channel blocking compound and the aspirin-like compound are co-administered.

41. The method of claim 40, wherein the sodium channel blocking compound and the aspirin-like compound are each co-administered using the same route of administration.

42. The method of claim 41, wherein the route of administration is by injection.

43. The method of claim 42, wherein injection is an intramuscular injection.

44. The method of claim 39, wherein the subject is experiencing acute pain.

45. The method of claim 39, wherein the subject is experiencing chronic pain.

46. The method of claim 20, wherein the aspirin-like compound is selected from the group consisting of salicylic acid or a salt thereof, acetylsalicylic acid, and diflunisal.

47. The method of claim 46, wherein the sodium channel blocking compound and the aspirin-like compound are co-administered.

48. The method of claim 47, wherein the co-administration of the sodium channel blocking compound and the aspirin-like compound results in a synergistic effect whereby improved analgesia is achieved in said subject as compared to analgesic effect of either the sodium channel blocking compound or the aspirin-like compound administered in the absence of the other compound.

49. The method of claim 48, wherein the sodium channel blocking compound and the aspirin-like compound are each co-administered using the same route of administration.

50. The method of claim 49, wherein the route of administration is by injection.

51. The method of claim 50, wherein injection is an intramuscular injection.

52. The method of claim 48, wherein the subject is experiencing acute pain.

53. The method of claim 48, wherein the subject is experiencing chronic pain.

54. The method of claim 29, wherein the aspirin-like is selected from the group consisting of salicylic acid or a salt thereof, acetylsalicylic acid, and diflunisal.

55. The method of claim 54, wherein the sodium channel blocking compound and the aspirin-like compound are co-administered.

56. The method of claim 55, wherein the co-administration of the sodium channel blocking compound and the aspirin-like compound results in a synergistic effect whereby improved analgesia is achieved in said subject as compared to analgesic effect of either the sodium channel blocking compound or the aspirin-like compound administered in the absence of the other compound.

57. The method of claim 56, wherein the sodium channel blocking compound and the aspirin-like compound are each co-administered using the same route of administration.

58. The method of claim 57, wherein the route of administration is by injection.

59. The method of claim 58, wherein injection is an intramuscular injection.

60. The method of claim 56, wherein the subject is experiencing acute pain.

61. The method of claim 56, wherein the subject is experiencing chronic pain.

62. A method for producing analgesia in a subject experiencing pain, the method comprising:
a. administering tetrodotoxin to the subject; and
b. administering aspirin to the subject;
wherein the administration of tetrodotoxin and aspirin to the subject is effective in producing analgesia in said subject.

63. The method of claim 62, wherein the tetrodotoxin and the aspirin are co-administered.

64. The method of claim 63, wherein the co-administration of tetrodotoxin and aspirin results in a synergistic effect whereby improved analgesia is achieved in said subject when compared to the analgesic effect of either tetrodotoxin or aspirin administered in the absence of the other compound.

65. The method of claim 64 wherein the tetrodotoxin and the aspirin are each co-administered using the same route of administration.

66. The method of claim 65, wherein the route of administration is by injection.

67. The method of claim 66, wherein injection is an intramuscular injection.

68. The method of claim 64, wherein the subject is experiencing acute pain.

69. The method of claim 64, wherein the subject is experiencing chronic pain.
